(19) United States
(12) Patent Application Publication
(10) Pub. No.: US 2026/0144530 A1
(43) Pub. Date: May 28, 2026

(54) SUSTAINABLE MEDICAL DEVICES, SYSTEMS, AND RELATED METHODS

(71) Applicant: Boston Scientific Scimed, Inc., Maple Grove, MN (US)

(72) Inventors: Juan Pablo ORTIZ GARCIA, Heredia (CR); Ryan V. WALES, Shrewsbury, MA (US); Alejandra GOMEZ JARA, Alajuela (CR)

(73) Assignee: Boston Scientific Scimed, Inc., Maple Grove, MN (US)

(21) Appl. No.: 19/399,769

(22) Filed: Nov. 25, 2025

Related U.S. Application Data
(60) Provisional application No. 63/725,592, filed on Nov. 27, 2024.

(51) Int. Cl. A61B 10/04; A61B 10/02; A61B 10/06; A61L 31/06

(52) U.S. Cl. CPC A61B 10/04; A61B 10/06; A61L 31/06; A61B 2010/0208

(57) ABSTRACT

Medical devices, systems, and related methods described herein may be useful for disassembling a medical device after performing a medical procedure to dispose of one or more portions of the medical device. The medical device including a shaft that includes a sheath having an outer wall that defines a lumen, a coil disposed within the sheath, and an end effector coupled to a distal portion of the shaft. The outer wall of the sheath includes at least one perforation configured to separate the sheath from the coil for disposal after performing the medical procedure.

### CROSS-REFERENCE TO RELATED APPLICATION(S)

This application claims the benefit of priority of U.S. Provisional Patent Application No. 63/725,592 , filed on Nov. 27, 2024, the entirety of which is incorporated herein by reference.

### TECHNICAL FIELD

Various aspects of the disclosure relate generally to sustainable medical devices, systems, and related methods. Examples of the disclosure relate medical instruments that may be partially disassembled following use to separately dispose of their elements.

### BACKGROUND

Single-use medical devices are disposed after use, resulting in significant medical waste and negative environmental impact. These medical devices often include a handle, a shaft, and an end effector configured to perform a procedure at a target site, such as biopsy procedures including the removal and analysis of a body tissue sample (“biopsy sample”) for medical diagnosis. It would be useful to improve medical devices, systems, and related methods for medical procedures, for example to reduce medical waste and environment impact associated with biopsy medical procedures.

### SUMMARY

Examples of this disclosure relate to systems, devices, and related methods for biopsy, among other things. For example, this disclosure describes examples that relate to shafts for biopsy forceps devices, among other aspects.

According to one aspect, the disclosure provides a medical device including a shaft and an end effector coupled to a distal portion of the shaft. The shaft including an outer wall defining a lumen; and a coil disposed within the lumen of the sheath. The outer wall of the sheath includes at least one perforation, and wherein the at least one perforation facilitates separation of the sheath from the coil so that the sheath and the coil may be separately disposed of after a procedure using the medical device.

According to some aspects, the at least one perforation may include at least two perforations that define at least one strip extending along at least one portion of the outer wall. The at least one perforation may include a plurality of cuts or openings that extend at least partially through the outer wall. The at least one strip may extend substantially parallel to a central longitudinal axis of the sheath. The at least one strip may include at least one tab configured to be gripped by a user to separate the at least one strip from a remainder of the sheath. The at least one tab may be positioned adjacent a proximal end of the sheath. The at least one tab may be positioned adjacent a distal end of the sheath. In some aspects, the at least one strip may include a plurality of strips arranged circumferentially around the outer wall. The plurality of strips may include a first strip and a second strip, wherein the first strip may be positioned diametrically opposite to the second strip. The first strip may be longitudinally offset from the second strip in a proximal or distal direction. In other aspects, the at least one strip may extend helically around the sheath.

According to some aspects, a distalmost end of the sheath may be proximal of a distalmost end of the coil. The sheath may include at least one of polylactic acid (PLA) or polybutylene adipate terephthalate (PBAT). The at least one perforation may include two perforations that define a strip extending along a portion of the outer wall, wherein the strip includes one of PLA or PBAT, and wherein a remainder of outer wall includes a different material. The medical device may further include a handle, and wherein the handle may include at least one of PLA or PBAT.

According to another aspect, the disclosure provides a medical device including a sheath that includes an outer wall defining a lumen, and a coil disposed within the lumen of the sheath. The outer wall may have at least two perforations that define a strip, wherein the strip is removable from a remainder of the outer wall to facilitate separation of the sheath from the coil so that the sheath and the coil may be separately disposed of after a procedure using the medical device.

According to some aspects, the strip may include at least one tab that is configured to be gripped by an operator for tearing away the strip from the remainder of the outer wall of the sheath. The strip may extend along an entire length of the sheath, and wherein the at least one tab may include a proximal tab at a proximal end of the sheath and a distal tab at a distal end of the sheath.

According to yet another aspect, the disclosure provides a medical device including a shaft, an end effector coupled to a distal portion of the shaft, and a handle coupled to a proximal portion of the shaft. The shaft including a sheath that includes an outer wall defining a lumen; and an actuation wire movably disposed within the lumen of the sheath, wherein the sheath includes at least one of polylactic acid (PLA) or polybutylene adipate terephthalate (PBAT).

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and, together with the description, serve to explain the principles of the disclosure.

FIG. 1 depicts an exemplary medical device.

FIG. 2 depicts another medical device.

FIG. 3 depicts another medical device.

FIG. 4 depicts another medical device.

FIG. 5 depicts another medical device.

FIG. 6 depicts another medical device.

### DETAILED DESCRIPTION

Examples of the disclosure include devices, systems, and related methods for medical instruments that can be disassembled after use so that different portions may be disposed according to different protocols.

As used herein, the term “distal” refers to a portion farthest away from a user when introducing a device into a patient and the term “proximal” refers to a portion closest to the user when placing the device into the subject. Labeled arrows “P” and “D” indicate proximal and distal directions, respectively. The terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to system, devices, and methods for performing various medical procedures and/or treating portions of the biliary duct, large intestine, small intestine, cecum, esophagus, any other portion of the gastrointestinal tract, and/or any other suitable patient anatomy (collectively referred to herein as a “target site”). The devices disclosed herein may be entirely disposable or may include a combination of disposable and reusable devices. Reference will now be made in detail to examples of the disclosure described above and illustrated in the accompanying drawings. Wherever possible, the same reference numbers will be used throughout the drawings to refer to the same or like parts. Unless otherwise specified, aspects of different embodiments may be combined in any suitable manner.

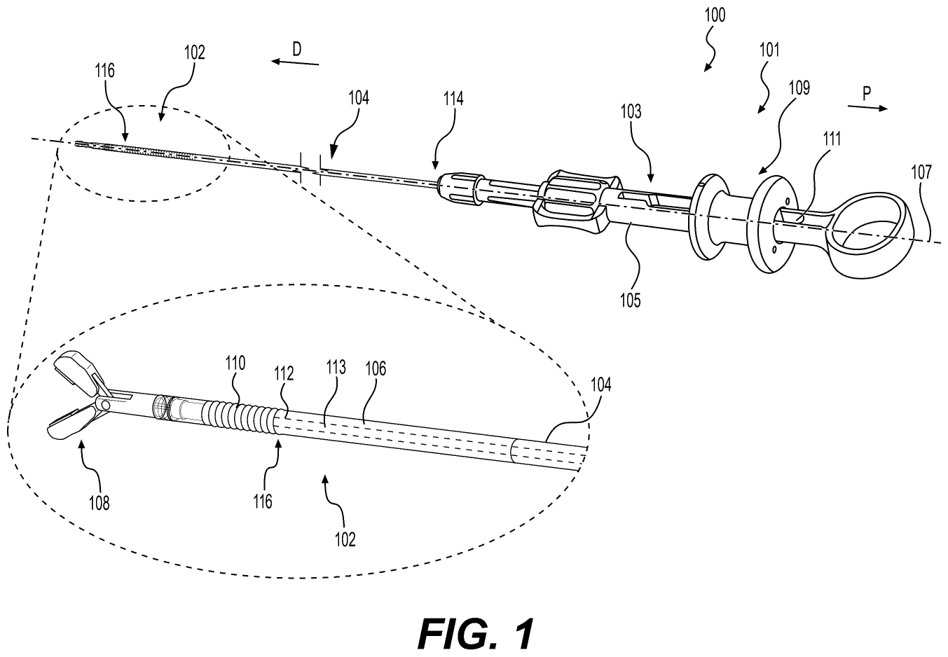

FIG. 1 illustrates a medical device 100 including a proximal portion 101, having a handle 103, and a distal portion 102, according to aspects of the present disclosure. Distal portion 102 may include a shaft 104. Shaft 104 may extend distally from handle 103 toward an end effector 108 of distal portion 102. End effector 108 may be positioned at a distal end of shaft 104. As shown in FIG. 1, end effector 108 may include a pair of jaws for, for example, extracting a biopsy sample of tissue from the target site. However, end effector 108 may be any suitable type of instrument (e.g., snare, stent delivery system, basket, balloon, knife, needle, clip, forceps, suture device, etc.). Although not shown, distal portion 102 may be delivered through a working channel or other lumen of an endoscope, duodenoscope, gastroscope, colonoscope, ureteroscope, bronchoscope, and/or other medical delivery systems to a target site.

Shaft 104 may include or otherwise radially surround a wire, for example, a pull wire or an actuation wire extending distally from handle 103 and through shaft 104. The wire(s) may be movable (e.g., within handle 103 and shaft 104) via manipulation of one or more portions of handle 103, for example one or more actuators 109 secured to a proximal end of the wire(s). End effector 108 may be coupled (e.g., directly or indirectly) to the distal end of the wire(s). In these aspects, manipulation of actuator 109 may control one or more aspects of medical device 100, for example, control one or more aspects of distal portion 102 such as end effector 108.

As shown, handle 103 includes a body 105 extending along a central longitudinal axis 107 of medical device 100. Body 105 may include one or more inner surfaces defining a lumen or channel of handle 103 which may, for example, be configured to receive or otherwise be coupled (e.g., directly or indirectly) to shaft 104.

Handle 103 may also include one or more movable members, for example actuator 109. Actuator 109 may be a spool, for example including an indented portion (e.g., with a relatively smaller lateral cross-section) and one or more (e.g., two) ridged or extended portions (e.g., flanges with a relatively larger lateral cross-section). In these aspects, the indented portion may receive one or more of a user's fingers, such that movement of the user's finger(s) controls the movement of actuator 109. Actuator 109 may be fixedly coupled to the actuation wire that controls end effector 108 or other aspects of distal portion 102. Although a spool is shown in FIG. 1, actuator 109 may alternatively be another type of actuator (e.g., finger rings, knob, lever, slider, joystick, etc.).

Body 105 may include a slot 111, for example, extending longitudinally through a portion of body 105, for example, from a position spaced distally from a ring of handle 103 (e.g., thumb ring). The ring may accommodate a thumb of a user's hand when the user engages actuator 109. A portion of actuator 109 may extend into a portion of slot 111, such that actuator 109 is movable (e.g., longitudinally movable distally and/or proximally) along slot 111. In these aspects, slot 111 may define a range of movement for actuator 109. Actuator 109 may be movable within slot 111, for example, to control one or more aspects of distal portion 102, such as to control end effector 108.

It should be understood that actuator 109 may be formed in a variety of sizes to accommodate users with different sized hands or fingers and/or to accommodate different needs of different users. In some examples, handle 103 includes two or more portions coupled together to define actuator 109. For example, actuator 109 may be split longitudinally into a first portion and a second portion, which are coupled together at complementary surfaces to form actuator 109. In other examples handle 103 may be unitary or integrally formed, for example by using one or more additive manufacturing and/or subtractive manufacturing processes to define the shape and dimensions of handle 103 and components thereof (e.g., body 105, actuator 109).

Shaft 104 may include a sheath 106 defining a lumen extending therein. Sheath 106 may include a non-metallic material, such as plastic (e.g., thermoplastic elastomer (TPE) or thermoplastic polyurethane (TPU), or thermoplastic polyesters), polyamide (e.g., nylon or Pebax), polytetrafluoroethylene (PTFE), or any other suitable non-metal (e.g., insulating) material. A distalmost end of sheath 106 may be directly adjacent to end effector 108. In such examples, FIG. 1 depicts a tear-away view such that portions of shaft 104 internal to sheath 106 are visible at a distal end of shaft 104. Alternatively, sheath 106 may have a distalmost end that terminates proximally of end effector 108, such that a non-zero distance is formed between the distalmost end of sheath 106 and end effector 108. For example, as described in further detail below, a portion of a coil 110 may extend distally of a distalmost end of sheath 106.

Shaft 104 may include one or more elements extending through the lumen of sheath 106, such as an actuation wire (discussed above) or coil 110 having a distal portion coupled to end effector 108 and a proximal portion coupled to handle 103. For coil 110 may be disposed in the lumen of sheath 106. Sheath 106 may be directly abutting and contacting coil 110. Coil 110 may be made of a metallic material (e.g., stainless steel, Nitinol, or any other suitable material) and may comprise a wire (having a round or other cross-sectional shape) that is wound about a mandrel or other structure to form a plurality of windings. Adjacent windings of coil 110 may contact one another, so that coil 110 has an outer wall without substantial gaps. Coil 110 may be shaped and sized to extend through the lumen of sheath 106, such that, and coil 110 may define a channel or lumen extending along a central axis coaxial or parallel with longitudinal axis 107. The channel of coil 110 may be configured to accommodate one or more actuation or pull wires for, for example, actuating end effector 108.

In some aspects, shaft 104 includes one or more features configured to facilitate disassembly of shaft 104, for example to separate various components having recyclable material and/or reusable material. Shaft 104 may be disassembled following use of device 100, and the separated components may be disposed of separately. For example, shaft 104 may include one or more biodegradable plastics or recyclable plastic material (e.g., to recycled and/or reused after the medical procedure). Coil 110, the actuation wire, and end effector 108 may all be formed of a metal that may be recyclable as a unit. In some examples, coil 110 may be removed from handle 103 by, for example, cutting coil 110 at its proximal end, where it is coupled to handle 103. In other examples, handle 103 may be recyclable along with coil 110 and end effector 108 (e.g., handle 103 may be formed of metal).

In conventional medical instruments it may be difficult and time consuming to separate a cover sheath from an internal coil. To facilitate such separation, sheath 106 may include one or more perforated portions having at least one perforation configured to facilitate separating sheath 106 and coil 110 disposed therein (e.g., after the medical procedure). As discussed herein, the perforated portion(s) may be arranged in various patterns or configurations, which may assist the user in removing one or more portions of the outer wall of sheath 106 (e.g., from around coil 110 received therein), thus supports disassembly of medical device 100 (e.g., to recycle, reuse, or otherwise dispose of one or more aspects of shaft 104).

In some examples, sheath 106 includes a perforation 112 extending along an outer wall of sheath 106, such that perforation 112 extends approximately parallel with longitudinal axis 107. Perforation 112 may include a series of cuts or openings that are formed in longitudinal succession with one another. Perforation 112 may be useful, for example, to facilitate separating sheath 106 from coil 110 disposed therein.

In some aspects, perforation 112 may be arranged in a pattern, combination of patterns, or otherwise define a perforated profile formed on one or more portions of outer wall of sheath 106. In the example shown in FIG. 1, sheath 106 includes two perforations 112, which may extend substantially parallel to one another. Perforations 112 may define a strip 113 between perforations 112 (e.g., defined by a closest distance between opposing perforations 112) extending between a proximal end 114 and a distal end 116 of sheath 106. Strip 113 has a substantially elongated rectangular profile along a longitudinal extent thereof, between proximal end 114 and distal end 116. In some examples, one or more of the cuts or openings of perforation 112 may have a rounded or curved shape, which may be useful to facilitate removal of perforation 112. For example, some or all of the openings of perforation 112 may have a scalloped shape.

In some aspects, perforation(s) 112 (e.g., two parallel perforations 112, as shown) may define one or more tabs of strip 113, for example at proximal end 114 and/or distal end 116 to support tearing or otherwise removing strip 113 from sheath 106. The tabs may be configured to be gripped by a user to separate strip 113 from sheath 106. For example, strip 113 may include a proximal tab at proximal end 114 and/or a distal tab at distal end 116. A proximal tab may extend proximally beyond adjacent portions of proximal end 114. A distal tab may extend distally beyond adjacent portions of distal end 116. In some examples, the tab(s) may be positioned adjacent to one or more ends of sheath 106, such that the user may grasp at least one tab for tearing strip 113 away from sheath 106 by moving at least one tab toward the opposing end or tab of strip 113.

In other implementations, perforation 112 may have a different perforated profile or combination of profiles. For example, perforation 112 may define strip 113 arranged in a spiral or helical pattern that winds around longitudinal axis 107. In some examples, perforation 112 is defined by cuts or slits having geometric shapes (e.g., hexagons, triangles, squares, circles, octagons, trapezoids, etc.) that collectively define the perforated profile of perforation 112. For instance, perforation 112 may include a plurality of diamond shaped cuts or openings arranged on sheath 106. The size and/or number of cuts or slits of each shape may be determined based on material strength of sheath 106 For example, properties of perforation 112 may be chosen so as to make strip 113 easier or harder to detach from a remainder of sheath 106, based on a material, shape, size, or intended use of sheath 106.

Disassembling medical device 100 (e.g., after the medical procedure) may include tearing strip 113 away from sheath 106 via perforations 112, for example by tearing strip 113 from proximal end 114 toward distal end 116 and/or tearing strip 113 from distal end 116 toward proximal end 114. This may allow the medical professional to separate (remove) a remainder of sheath 106 from coil 110. Removing strip 113 may define one or more voids or gaps in the outer wall of sheath 106, which may be useful for example to facilitate disassembling medical device 100 by peeling away the remainder of sheath 106 from coil 110. Sheath 106 having a distalmost end that is proximal of a distalmost end of coil 110 may facilitate tearing off strip 113 and separating sheath 106 from coil 110. In some examples, an operator may cut coil 110 to separate it from handle 103 to further facilitate disposal (e.g., recycling) of coil 110 and end effector 108.

In some aspects, sheath 106 may include one or more plastic materials, such as a thermoplastic or biodegradable plastic. For example, sheath 106 may include one or more biomaterials, such as polylactic acid (PLA) or polybutylene adipate terephthalate (PBAT). In some examples, perforation 112 may be formed by mechanical deformation or weakening the plastic material(s) of sheath 106. For instance, mechanically deforming the outer wall substantially along a length of sheath 106 may define perforation 112 thereon. In some implementations, the outer wall of sheath 106 includes a first material and strip 113 includes a second material, different from the first material.

In some implementations, sheath 106 includes a biodegradable material (e.g., a plurality of biodegradable layers. Forming these biodegradable layers may include, for example, successive deposition of biodegradable plastic (e.g., extrusion) to form sheath 106 having a thickness of the outer wall being defined by size or shape of the deposited layers. In these examples, forming perforation 112 may include subtractive manufacturing or other processes (e.g., CNC machining, milling, lathing, etc.) that deform the outer wall and define perforation 112 thereon. For instance, perforation 112 may be formed by laser etching or engraving the perforated pattern(s) on the outer wall of sheath 106 by exposing sheath 106 to energy which causes mechanical deformation in the pattern(s), thereby yielding perforation 112. Alternatively, perforation 112 may be created during an additive manufacturing procedure of forming sheath 106.

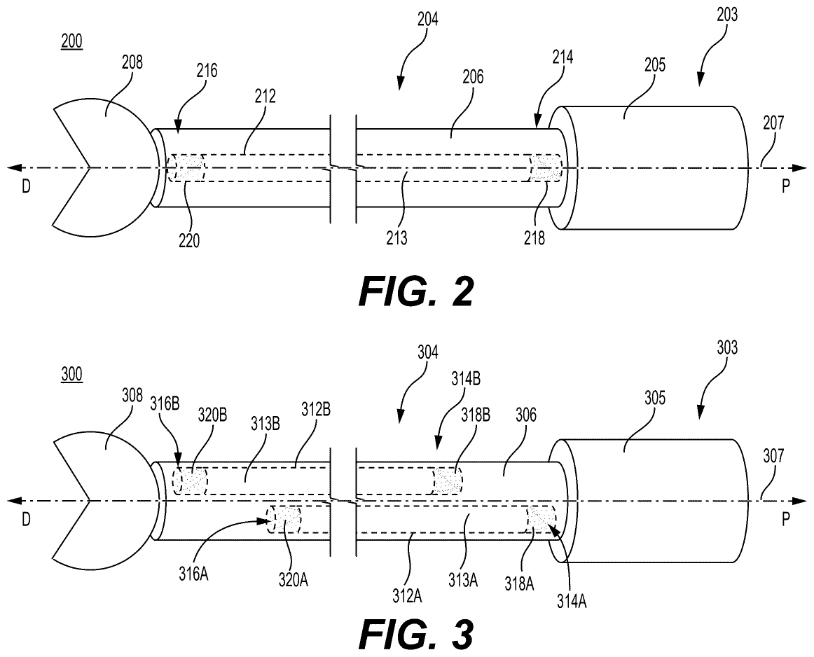

FIG. 2 illustrates another medical device 200, including a shaft 204 that is attached a handle 203 to perform a medical procedure, according to aspects of the disclosure. Medical device 200 may include one or more aspects of medical device 100, unless otherwise specified. As shown, shaft 204 includes a sheath 206 having an outer wall defining a lumen therein, sheath 206 extending from handle 203 to a region adjacent an end effector 208. Shaft 204 may include a coil (not shown but having any of the properties of coil 110) extending through the lumen of sheath 206. A proximal portion of the coil may be coupled with handle 203 and a distal portion of the coil may be coupled with end effector 208. Handle 203 includes a body 205. As discussed above, in some examples, the coil may be separated (e.g., cut) from body 205 to disassemble medical device 200 prior to disposal. As discussed herein, shaft 204 includes one or more aspects that support disassembly of medical device 200, for example to support separation of sheath 206 from the coil therein.

Shaft 204 also includes at least one perforation 212 (having any of the properties of perforation 112, unless otherwise specified) extending along an extent of the outer wall of sheath 206. The at least one perforation 212 may include, for example, a plurality of cuts or openings that extend at least partially through the outer wall of sheath 206 along extent thereof. For example, as shown in FIG. 2, sheath 206 may include two perforations, which may extend substantially parallel to one another and define a strip 213. Strip 213 may include a proximal end 214 having a proximal tab 218 and a distal end 216 having a distal tab 220. In some implementations, strip 213 extends along an entire length of the outer wall of sheath 206.

Proximal tab 218 and/or distal tab 220 may be configured for helping an operator to tear strip 213 away a remainder of sheath 206, which leaves an opening or void along the length of sheath 206 to support separation of sheath 206 from the coil disposed therein. In some examples, proximal tab 218 and/or distal tab 220 may extend or protrude radially outward from strip 213 relative to a longitudinal axis 207 of medical device 200, which may facilitate the user grasping proximal tab 218 and/or distal tab 220 for tearing (e.g., proximally and/or distally) strip 213 away from the outer wall of sheath 206. In examples, tab 218 may extend further proximally than adjacent portions of sheath 206, and tab 220 may extend further distally than adjacent portions of sheath 206. In aspects, tab 218 and tab 220 may be defined by a solid (non-perforated) cut to separate tabs 218 and 220 from surrounding portions of sheath 206. In some implementations, proximal tab 218 and/or distal tab 220 may have a hook, latch or other mechanism protruding radially outward relative to the longitudinal axis 207 (e.g., to facilitate gripping/grasping/moving/tearing of strip 213 after the medical procedure).

In these aspects, the disclosure provides a method that may include the steps of: (i) performing the medical procedure with medical device 200, for example performing a biopsy procedure for extracting biopsy sample of tissue from a target site of a subject using end effector 208; (ii) optionally detaching shaft 204 from handle 203 (e.g., by cutting); (iii) grasping at least one of proximal tab 218 or distal tab 220 to tear strip 213 away from sheath 206; and (iv) separating the remaining portions of sheath 206 from one or more components received therein, such as the coil disposed therein. In some examples, steps (ii) and (iii) may be reversed in order.

As shown, proximal tab 218 is positioned adjacent a proximal end of sheath 206 adjacent to handle 203, and distal tab 220 is positioned adjacent or otherwise proximate to end effector 208 at distal end of sheath 206. However it should be understood that different arrangements or other configurations of perforation 212 and strip 213 are contemplated herein. For example, perforation 212 may extend diagonally across sheath 206, such that strip 213 extends from proximal end 214 to distal end 216 along a central axis oriented at an angle relative to longitudinal axis 207. In other examples, as shown, perforation 212 extends approximately parallel with longitudinal axis 207, such that strip 213 extends from proximal end 214 to distal end 216 along the central axis approximately parallel with longitudinal axis 207. In some implementations, the outer wall of sheath 206 includes a first material and strip 213 includes two or more materials, different from the first material.

FIG. 3 illustrates another medical device 300, including a shaft 304 that is attached a handle 303 to perform a medical procedure, according to aspects of the disclosure. Unless otherwise specified, medical device 300 may include one or more aspects of medical device 100 and/or medical device 200. As shown, shaft 304 includes a sheath 306 having an outer wall defining a lumen therein, sheath 306 extending from handle 303 to a region adjacent an end effector 308. Shaft 304 may include a coil (not shown but having any property of coil 110) extending through the lumen of sheath 306. A proximal portion of the coil may be coupled with handle 303 and a distal portion of the coil may be coupled with end effector 308. Handle 303 includes a body 305. In some examples, the coil may be separated from body 305 to disassemble medical device 300 prior to disposal. As discussed herein, shaft 304 includes one or more aspects that support disassembly of medical device 300, for example to support separation of sheath 306 from the coil therein.

Shaft 304 also includes at least one perforation (e.g., plurality of perforations) extending along an extent of the outer wall of sheath 306 (e.g., having any property of perforation 112 and/or perforation 212, unless otherwise specified). For example, shaft 304 may include a pair of proximal perforations 312A that defines a first strip 313A and a pair of distal perforations 312B that defines a second strip 313B, each strip 313A-313B extending along outer wall of sheath 306 (e.g., substantially parallel with a longitudinal axis 307). First strip 313A may extend from a first proximal end 314A having a first proximal tab 318A and to a first distal end 316A having a first distal tab 320A, e.g., along the first extent of the outer wall of sheath 306 (e.g., proximal portion). Second strip 313B may extend from a second proximal end 314B having a second proximal tab 318B and to a second distal end 316B having a second distal tab 320B, e.g., along the second extent of the outer wall of sheath 306 (e.g., distal portion). As such, shaft 304 includes a plurality of strips (e.g., first strip 313A and second strip 313A) at a plurality of positions (e.g., proximal position and distal position) along the outer wall of sheath 306.

In some example, first strip 313A may be circumferentially aligned with second strip 313B. In alternatives, first strip 313A and second strip 313A may be longitudinally offset from one another, such that first strip 313A is longitudinally offset from second strip 313B in a proximal direction or distal direction relative to a longitudinal axis 307. As shown, first distal end 316A may be proximal of second proximal end 314B, such that strips 313B and 313A overlap one another longitudinally. In other examples, second proximal end 314B may be distal of first distal end 316A. In aspects, each of first strip 313A and second strip 313B may extend at a same angle relative to a longitudinal axis 307 (e.g., parallel to longitudinal axis 307). In alternatives, first strip 313A and second strip 313B may have different, non-zero angles with respect to longitudinal axis 307. It should be understood that shaft 304 may have the plurality of perforations in different arrangements or combinations, such as relative spacing between proximal perforation 312A and distal perforation 312B. Similarly, the plurality of perforations may be arranged or otherwise configured to have different shapes, sizes, or quantity. For example, medical device 300 may have three, four, five, six, seven, eight or more perforated strips extending on outer wall of sheath 306.

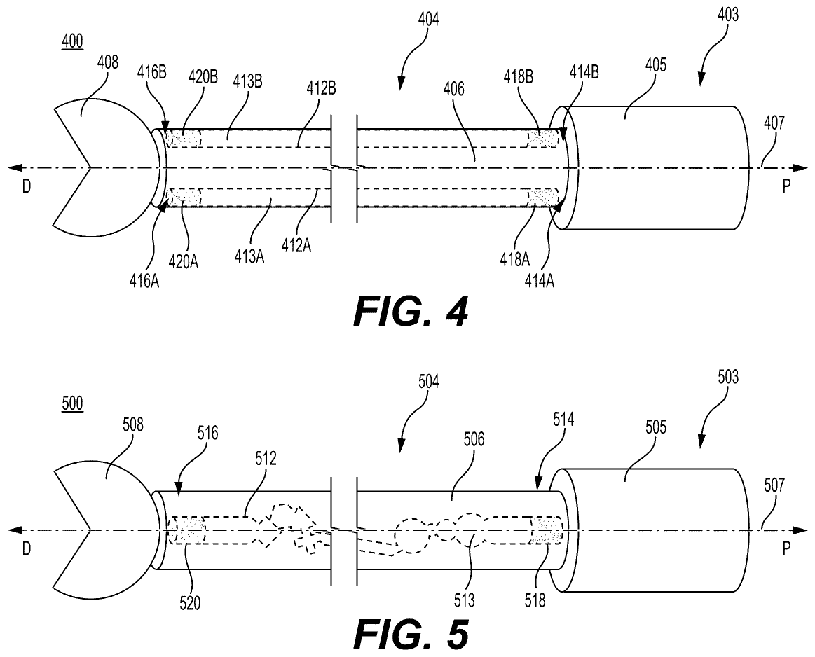

FIG. 4 illustrates another medical device 400, including a shaft 404 that is selectively attachable to a body 405 of a handle 403. Unless otherwise specified, medical device 400 may include one or more aspects of medical devices 100, 200, and/or 300. As shown, shaft 404 includes a sheath 406 having an outer wall defining a lumen therein, and which extends from a proximal end of sheath 406 attachable to handle 403 toward an end effector 408 of shaft 404 adjacent to a distal end of sheath 406. Shaft 404 may include a coil extending through the lumen of sheath 406, for example such that a proximal portion of the coil may be coupled with handle 403 (e.g., at proximal end of shaft 404) and a distal portion of coil may be coupled with end effector 408 (e.g., at distal end of shaft 404).

Shaft 404 may also include a first perforation 412A and a second perforation 412B, each of perforations 412A, 412B extend along respective extents of the outer wall of sheath 406 relative to a central longitudinal axis 407 of medical device 400. In the example shown, a pair of first perforations 412A defines a first strip 413A including a first proximal end 414A having a first proximal tab 418A and a first distal end 416A having a first distal tab 420A, and a pair of second perforations 412B defines a second strip 413B including a second proximal end 414B having a second proximal tab 418B and a second distal end 416B having a second distal tab 420B.

In other implementations, perforations 412A-412B may have different perforated profiles or combinations of profiles. For example, perforations 412A-412B may be arranged such that first strip 413A extends at a first position and second strip 413B extends at a second position relative to the central axis 407 different than the first position. First strip 413A may be offset circumferentially from second strip 413B relative to central axis 407. In another example, first strip 413A may be positioned diametrically opposite or opposed to second strip 413B, e.g., along the outer wall of sheath 406 relative to central axis 407. In yet another example, shaft 404 may include four (4) perforated strips formed on the outer wall of sheath 406, such that each perforated strip is separated by approximately ninety degrees (90°) away from adjacent strips in either a first direction or a second direction (e.g., clockwise or counter-clockwise) relative to central axis 407 about the outer wall of sheath 406. As shown in FIG. 4, each of perforations 412A, 412B may extend along an entire length of sheath 406; alternatively, one or more of perforations 412A, 412B may extend along only a portion of a longitudinal length of sheath 406.

As discussed above, removing strips 413A and 413B aids in removing portions of the outer wall of sheath 406 which remain after strips 413A and 413B are removed. (e.g., disposed around the coil received therein). The user may leverage openings or voids that are formed along the outer wall of sheath 406 (e.g., formed due to removing/tearing strips 413A and 413B from a remainder of sheath 406).

FIG. 5 illustrates another medical device 500, including a shaft 504 that is attached a handle 503 to perform a medical procedure, according to aspects of the disclosure. Medical device 500 may include any aspect of medical devices 100, 200, 300 and/or 400, unless otherwise specified. Shaft 504 includes a sheath 506 having an outer wall defining a lumen therein, sheath 506 extending from handle 503 to a region adjacent an end effector 508. Shaft 504 may include a coil (not shown but having any of the properties of coil 110) extending through the lumen of sheath 506, for example the coil may include a proximal portion coupled with handle 503 and a distal portion coupled with end effector 508. The coil may be separated from a body 505 of handle 503 to disassemble medical device 500 prior to disposal. As discussed herein, shaft 504 includes one or more aspects that support disassembly of medical device 500, for example to support separation of sheath 506 from the coil therein.

Shaft 504 also includes perforations 512 extending along an extent of the outer wall of sheath 506 and defining a strip 513 thereon. Strip 513 may include a proximal end 514 having a proximal tab 518 and a distal end 516 having a distal tab 520. Proximal tab 518 and/or distal tab 520 may facilitate tearing strip 513 away a remainder of sheath 506, and in turn support separation of sheath 506 from the coil disposed therein. Strip 513 may be defined by a plurality of perforated portions (e.g., perforations 512) having various geometric shapes which are interconnected along an extent of strip 513 (e.g., bound between proximal end 514 and distal end 516). In the example shown, strip 513 includes shapes sharing one or more sides. The shapes may include but are not limited to squares, trapezoids, triangles, crosses, diamonds, circles, and ovals. Varying the shape and size of strip 513 along the extent of sheath 506 may adjust the ease with which strip 513 may be removed and/or a durability of medical device 500 during the medical procedure (e.g., resistance to stress and strain). Increasing the quantity and/or size of perforations 512 may decrease force required to tear sheath 506 or otherwise disassemble shaft 504 after the medical procedure.

In some examples, as shown in FIG. 5, opposing perforations 512 that define strip 513 may extend along a path that is generally parallel to one another. As shown, perforations 512 may be mirror images of one another across an axis extending down a center of strip 513. Alternatively, perforations 512 may be parallel along their length.

In some aspects, a medical system may include one or more medical devices, for example as discussed herein. For example, the medical system may include medical device 100, 200, 300, 400, and/or 500. The medical system may also include a medical tool configured to disassemble the disposable portion of said medical device, for example to facilitate separation of at least one sheath from at least one coil disposed therein. In some implementations, the medical tool includes a cutting tool that is configured to cut or tear one or more perforations or perforated portions of the medical device, for example to facilitate separation of a perforated strip defined by these perforated portions (e.g., without tabs at ends thereof). The medical tool may also be useful for cutting implementations of the medical device without any, or having very little, perforations thereon. For example, a sheath formed of a biodegradable plastic or recyclable plastic having material properties that support cutting of the sheath with the medical tool, such as pliers or shears.

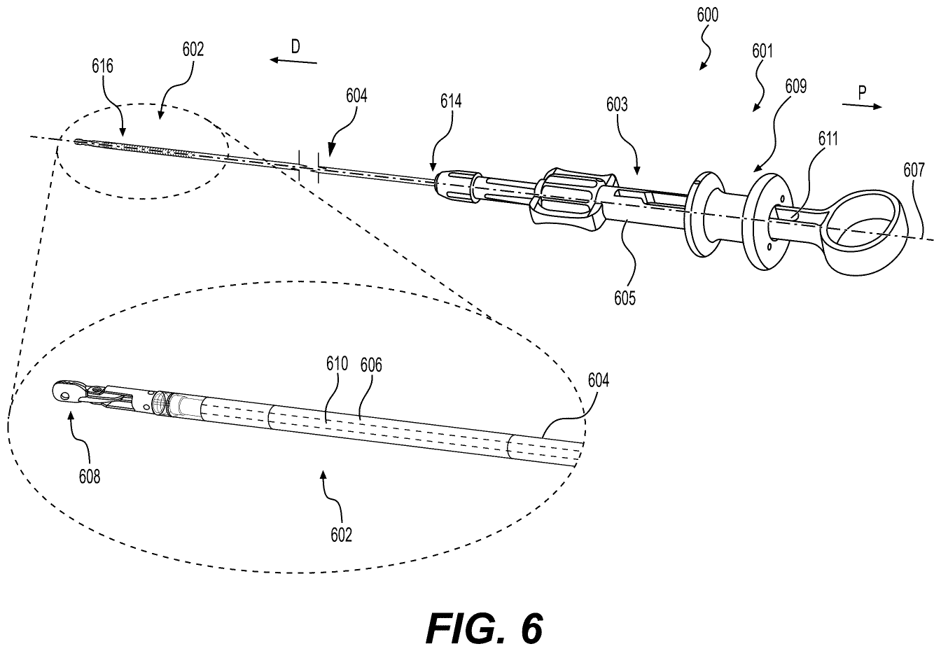

FIG. 6 illustrates another medical device 600 including a proximal portion 601 having a handle 603 and a distal portion 602 having a shaft 604, according to aspects of the disclosure. Unless otherwise specified, medical device 600 may include any aspect of medical devices 100, 200, 300, 400 and/or 500. As shown, shaft 604 includes a sheath 606 having an outer wall defining a lumen therein, e.g., sheath 606 extending distally from handle 603 toward an end effector 608 of distal portion 602, e.g., a medical instrument positioned at a distal end of shaft 604, e.g., pair of jaws for extracting a biopsy sample of tissue from a target site within a subject. Shaft 604 may include one or more elements extending through the lumen of sheath 606, such as an actuation wire 610 extending distally from handle 603 through the lumen to a distal end which, for example, may be coupled (e.g., directly or indirectly) to end effector 608.

Handle 603 includes a body 605 extending along a central longitudinal axis 607 of medical device 600. Body 605 may include inner surfaces defining a lumen or channel of handle 103, for example configured to be coupled with shaft 604 and receive actuation wire 610 therethrough. Handle 603 also includes one or more movable members attachable to actuation wire 610, for example an actuator 609 (e.g., a spool) movably coupled to a proximal portion of actuation wire 610 for controlling end effector 608. Actuator 609 may extend at least partly into a portion of a slot 611 formed in body 605, such that actuator 609 is movable (e.g., distally and/or proximally) along slot 611 to control one or more aspects of distal portion 602 (e.g., end effector 608).

In some aspects, sheath 606 may include one or more plastic materials, such as a thermoplastic or biodegradable plastic. For example, sheath 606 may include one or more biomaterials, such as PLA and/or PBAT. Handle 603 may also include one or more plastic materials, such as a thermoplastic or biodegradable plastic. For example, one or more components of handle 603 (e.g., body 605, actuator 609) may include PLA and/or PBAT. This may be useful for recycling, composting or otherwise disposing of shaft 604 and/or handle 603, e.g., to mitigate environmental impact and reduce medical waste as a result of performing the medical procedure.

In some implementations, shaft 604 also includes at least two perforations that define at least one strip extending along at least one portion of the outer wall of sheath 606. Although not shown, for example, shaft 604 may have one or more perforations 112, 212, 312A-312B, 412A-412B, and/or 512, respectively defining one or more strips 113, 213, 313A-313B, 413A-413B, and/or 513, extending along one or more extents of the outer wall of sheath 606.

Medical devices, systems, and related methods discussed herein may be useful for performing biopsy medical procedures to acquire a biopsy sample. Disassembling medical devices described herein may include tearing one or more perforations of one or more disposable portions, for example to separate a sheath from a coil disposed therein. This may be useful, for example, to quickly disassemble the medical device (e.g., after performing the medical procedure) for disposing the disposable portion (e.g., the sheath and/or the coil). The disposable portion may include biodegradable material or recyclable material, which may mitigate environmental impact associated with use of these devices.

Each of the aforementioned devices, systems, and methods may be used for medical procedures to acquire a biopsy sample from a patient. By providing a medical device capable of acquiring biopsy samples using a reusable handle assembly, known problems associated with biopsy medical procedures and/or other aspects of invasive surgical procedures may be reduced or avoided. Based on these aspects, physicians or other users of may reduce the overall procedure time, increase efficiency of procedures, and/or avoid unnecessary harm to patient anatomy during procedures that involve acquiring biopsy samples.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device, comprising:
a shaft including:
a sheath including an outer wall defining a lumen; and
a coil disposed within the lumen of the sheath; and
an end effector coupled to a distal portion of the shaft,
wherein the outer wall of the sheath includes at least one perforation, and
wherein the at least one perforation facilitates separation of the sheath from the coil so that the sheath and the coil may be separately disposed of after a procedure using the medical device.

2. The medical device of claim 1, wherein the at least one perforation includes at least two perforations that define at least one strip extending along at least one portion of the outer wall.

3. The medical device of claim 2, wherein the at least one strip extends substantially parallel to a central longitudinal axis of the sheath.

4. The medical device of claim 3, wherein the at least one strip includes at least one tab configured to be gripped by a user to separate the at least one strip from a remainder of the sheath.

5. The medical device of claim 4, wherein the at least one tab is positioned adjacent a proximal end of the sheath.

6. The medical device of claim 4, wherein the at least one tab is positioned adjacent a distal end of the sheath.

7. The medical device of claim 2, wherein the at least one strip comprises a plurality of strips arranged circumferentially around the outer wall.

8. The medical device of claim 7, wherein the plurality of strips includes a first strip and a second strip, wherein the first strip is positioned diametrically opposite to the second strip.

9. The medical device of claim 8, wherein the first strip is longitudinally offset from the second strip in a proximal or distal direction.

10. The medical device of claim 3, wherein the at least one strip extends helically around the sheath.

11. The medical device of claim 1, wherein a distalmost end of the sheath is proximal of a distalmost end of the coil.

12. The medical device of claim 1, wherein the at least one perforation includes a plurality of cuts or openings that extend at least partially through the outer wall.

13. The medical device of claim 1, wherein the sheath includes at least one of polylactic acid (PLA) or polybutylene adipate terephthalate (PBAT).

14. The medical device of claim 13, further comprising a handle, wherein the handle includes at least one of polylactic acid (PLA) or polybutylene adipate terephthalate (PBAT).

15. The medical device of claim 14, wherein the at least one perforation includes two perforations that define a strip extending along a portion of the outer wall, wherein the strip includes one of PLA or PBAT, and wherein a remainder of outer wall includes a different material.

16. A medical device, comprising:
a sheath, including an outer wall defining a lumen; and
a coil disposed within the lumen of the sheath; and
wherein the outer wall has at least two perforations that define a strip, wherein the strip is removable from a remainder of the outer wall to facilitate separation of the sheath from the coil so that the sheath and the coil may be separately disposed of after a procedure using the medical device.

17. The medical device of claim 16, wherein the strip includes at least one tab that is configured to be gripped by an operator for tearing away the strip from the remainder of the outer wall.

18. The medical device of claim 17, wherein the strip extends along an entire length of the sheath, and wherein the at least one tab includes a proximal tab at a proximal end of the sheath and a distal tab at a distal end of the sheath.

19. A medical device, comprising:
a shaft including:
a sheath including an outer wall defining a lumen; and
an actuation wire movably disposed within the lumen of the sheath;
an end effector coupled to a distal portion of the shaft; and
a handle coupled to a proximal portion of the shaft,
wherein the sheath includes at least one of polylactic acid (PLA) or polybutylene adipate terephthalate (PBAT).

20. The medical device of claim 19, wherein the handle includes at least one of polylactic acid (PLA) or polybutylene adipate terephthalate (PBAT).
